(19) United States
(12) Patent Application Publication
(10) Pub. No.: US 2021/0030402 A1
(43) Pub. Date: Feb. 4, 2021

(54) METHOD AND SYSTEM FOR PROVIDING REAL-TIME END OF ULTRASOUND EXAMINATION ANALYSIS AND REPORTING

(71) Applicant: GE Precision Healthcare LLC, Wauwatosa, WI (US)

(72) Inventors: Svein Arne Aase, Horten (NO); Eigil Samset, Oslo (NO)

(21) Appl. No.: 16/525,072

(22) Filed: Jul. 29, 2019

(51) Int. Cl. A61B 8/08; A61B 8/00; A61B 5/00

(52) U.S. Cl. CPC A61B 8/5276; A61B 8/469; A61B 5/7264; A61B 8/463; A61B 8/5246

(57) ABSTRACT

A system and method for performing an ultrasound examination, analyzing the acquired images, and reporting image inadequacies and missing views at the end of the ultrasound examination is provided. The method includes acquiring ultrasound images during an examination, each of the images having an image view. The method includes receiving a user input to end the examination. The method includes automatically determining, with artificial intelligence, whether one or more images include an inadequacy. The method includes automatically determining, with the artificial intelligence, whether desired image views are not present in the images. The method includes presenting a report identifying any inadequate images and missing views. The method includes providing a selectable reopen exam option to reopen the examination.

### FIELD

Certain embodiments relate to ultrasound imaging. More specifically, certain embodiments relate to a method and system for providing analysis and reporting of image inadequacies and missing views at an end of an ultrasound examination to provide an ultrasound operator an opportunity to reopen the examination to acquire additional ultrasound images prior to a patient leaving the examination.

### BACKGROUND

Ultrasound imaging is a medical imaging technique for imaging organs and soft tissues in a human body. Ultrasound imaging uses real time, non-invasive high frequency sound waves to produce a series of two-dimensional (2D) and/or three-dimensional (3D) images.

An ultrasound operator typically presses an “End Exam” button when the operator believes that all of the appropriate image views have been acquired. The patient is typically asked to get dressed and leave once the ultrasound examination is complete. If the acquired images are later determined to be inadequate and/or if needed image views are missing, the patient may be asked to return to undergo an additional ultrasound examination, which may be inconvenient, inefficient, and costly. Accordingly, ultrasound operators typically quickly and manually review the acquired ultrasound images to attempt to detect missing image views prior to the end of the examination. However, the manual review of the images is subjective, non-consistent, and error prone, particularly with an increasing number of recorded images. The effectiveness of the manual review approach is also limited to potentially detecting missing image views. Possible inadequacies with acquired ultrasound images, such as non-standard views, unintended heart rate variability, inconsistent anatomical structure dimensions in acquired images, and the like, may not be detected using the manual review approach.

For example, an ultrasound operator may have attempted to record a specific standard view but may be unsuccessful. The non-standard view of the acquired ultrasound image may be difficult to detect manually.

As another example, in regular echo examinations (not stress echo), the goal is to acquire all images at approximately a same heart rate to allow measurements calculated based on multiple images to evaluate the health of the heart at rest. However, a stress level of a patient may vary during the course of the examination due to discomfort, psychology, and/or random events. Additionally, extra systole or other rhythm disturbances could occur without the ultrasound operator noticing the disturbances (e.g., if an operator is recording multiple cycles). Also, problems may arise with an ECG signal quality and/or the QRS trig algorithm. The above exemplary factors may bias the resulting measurements and analysis without an ultrasound operator noticing at or before the end of the ultrasound examination (i.e., when new recordings could have been acquired).

Furthermore, inexperienced operators may have difficulty positioning the ultrasound probe for various examinations, which may result in inconsistent measured anatomical structure dimensions and/or fore-shortening. For example, if an ultrasound operator fails to properly position the ultrasound probe at an apical position when acquiring images of the apex of the heart, the imaged length of the left ventricle at end-diastole may be different from the imaged length at end-systole, which is referred to as fore-shortening. Measurements based on fore-shortened images may be wrong and/or misleading. As another example, if an ultrasound operator fails to properly position the ultrasound probe at the thickest part of the left ventricle when attempting to acquire a parasternal long axis (PLAX) view, a measured diameter of the left ventricle in the incorrectly acquired ultrasound image may be inaccurate and inconsistent with the diameter estimated in apical view images. Ultrasound images acquired at inaccurate probe positions may be difficult detect by a manual review of the acquired ultrasound images at the end of an ultrasound examination.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for analyzing and reporting image inadequacies and missing views at an end of an ultrasound examination, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to perform an ultrasound examination, analyze the acquired images, and report image inadequacies and missing views at the end of the ultrasound examination, in accordance with various embodiments.

FIG. 2 is a display of an exemplary ultrasound image examination report presented at the end of an ultrasound examination, in accordance with various embodiments.

FIG. 3 is a flow chart illustrating exemplary steps that may be utilized for performing an ultrasound examination, analyzing the acquired images, and reporting image inadequacies and missing views at the end of the ultrasound examination, in accordance with various embodiments.

### DETAILED DESCRIPTION

Certain embodiments may be found in a method and system for performing an ultrasound examination, analyzing the acquired images, and reporting image inadequacies and missing views at the end of the ultrasound examination. Various embodiments have the technical effect of identifying image inadequacies and missing views at an end of an ultrasound examination to provide an ultrasound operator an opportunity to reopen the examination to acquire additional ultrasound images prior to a patient leaving the examination.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams. Moreover, as used herein, the term “image” broadly refers to both single images and image loops (e.g., a recording of a plurality of still frames stored together).

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

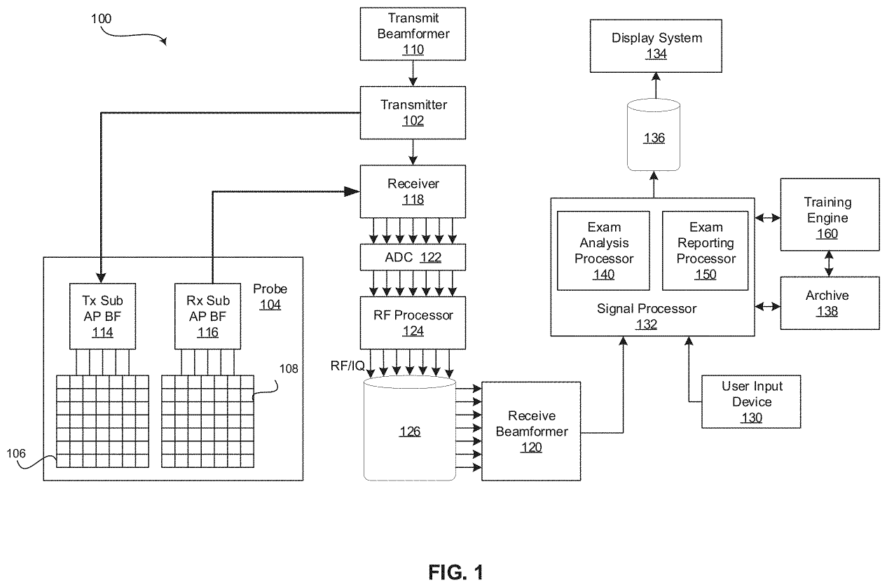

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to perform an ultrasound examination, analyze the acquired images, and report image inadequacies and missing views at the end of the ultrasound examination, in accordance with various embodiments. Referring to FIG. 1, there is shown an ultrasound system 100. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, an archive 138, and a training engine 160.

The transmitter 102 may comprise suitable logic, circuitry, interfaces and/or code that may be operable to drive an ultrasound probe 104. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. In certain embodiment, the ultrasound probe 104 may be operable to acquire ultrasound image data covering at least a substantial portion of an anatomy, such as the heart, a blood vessel, or any suitable anatomical structure.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, end an ultrasound examination, select an option to reopen the ultrasound examination or to confirm the end of the examination, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134, for example. As an example, user input device 130 may include a touchscreen display.

In various embodiments, an ultrasound examination is ended in response to a directive received via the user input device 130. A confirmation of the end of the examination or a directive to reopen the examination is subsequently received via the user input device 130 in response to an ultrasound image examination report presented at the end of an ultrasound examination at the display system 134. For example, an ultrasound operator may select an “End Exam” button of the user input device 130 on the ultrasound system probe 104, control panel, display system 134, or the like, once the operator believes that all the desired ultrasound images have been acquired. As described in more detail below, the signal processor 132 may analyze the acquired ultrasound images and provide a report summarizing the examination and identifying possible problems with the acquired images at the display system 134. The ultrasound operator may select to end the examination or reopen the examination to acquire additional images in response to the ultrasound image examination report via the user input device 130.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise an exam analysis processor 140 and an exam reporting processor 150 and may be capable of receiving input information from a user input device 130 and/or archive 138, generating an output displayable by a display system 134, and manipulating the output in response to input information from a user input device 130, among other things. The signal processor 132, exam analysis processor 140, and exam reporting processor 150 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include an exam analysis processor 140 that comprises suitable logic, circuitry, interfaces and/or code that may be operable to analyze acquired ultrasound images to determine whether the acquired views are adequate and whether any views are missing. The exam analysis processor 140 may include image analysis algorithms, one or more deep neural networks (e.g., a convolutional neural network) and/or may utilize any suitable form of image analysis techniques or machine learning processing functionality configured to automatically identify inadequate and missing views of an anatomical structure provided in the ultrasound image data. For example, the exam analysis processor 140 may include one or more analysis modules or algorithms such as, view recognition, spectrum recognition, end diastole estimation, segmentation, automated measurements (e.g., left ventricle (LV) study, cardiac automated Doppler, etc.), automated clinical findings (e.g., diastology assessment), and the like.

For example, view and spectrum recognition modules or algorithms may be configured to identify potential non-standard views if a view provided by an image is not recognized. The view and spectrum recognition modules or algorithms may be configured to identify missing image views, such as to perform automated functional imaging (AFI) analysis, American Society of Echocardiography (ASE) guideline measurements, or the like. As an example, if performing AFI analysis, the view and spectrum recognition modules or algorithms may identify if any of a four-chamber (4CH) view, a two-chamber (2CH) view, or an apical long-axis (APLAX) view is missing.

As another example, automated measurements and automated clinical findings modules or algorithms may be configured to identify images having inadequacies that may prevent subsequent automated measurements or other analysis. For example, the automated measurement module may attempt to execute automated measurements (e.g., an LV study on identified parasternal long-axis (PLAX) images or cardiac automated Doppler on identified spectrum images) and identify images that have a measurement confidence level below a pre-determined threshold.

Additionally, an end diastole estimation module or algorithm may be configured to generate a heart rate graph as a function of time during an ultrasound examination for all of the images in the examination. For example, the heart rate estimates may be extracted from ECG trig to ECG trig (QR-QR intervals) based on an electrocardiogram (ECG) and plotted against the exam times as provided for each image in the raw data/DICOM header.

Furthermore, a segmentation module or algorithm may be configured to identity dimension problems with imaged structures. For example, an artificial intelligence segmentation module or algorithm may be executed on the acquired ultrasound images. The segmentation module or algorithm may extract lengths and/or diameters, such as a length or diameter of a left ventricle in multiple ultrasound images at end diastole and end systole. The extracted lengths and/or diameters from the plurality of ultrasound images may be compared to identify outliers (e.g., inconsistent measurements). The existence of outliers may indicate probe misplacement during image acquisition with respect to one or more of the ultrasound images.

In various embodiments, any of the analysis modules or algorithms provided as a deep neural network executed by the exam analysis processor 140 may be made up of, for example, an input layer, an output layer, and one or more hidden layers in between the input and output layers. Each of the layers may be made up of a plurality of processing nodes that may be referred to as neurons. For example, an artificial intelligence view recognition analysis module or algorithm may include an input layer having a neuron for each pixel or a group of pixels from a scan plane of an anatomical structure. The output layer may have a neuron corresponding to a plurality of pre-defined views. As an example, if imaging a heart, the output layer may include neurons for a 4CH view, a 2CH view, an APLAX view, a PLAX view, a short-axis apical level (SAX-AP) view, a short-axis papillary muscle level (SAX-PM) view, a short-axis mitral valve level (SAX-MV) view, an unknown view, an other view, and/or any suitable view. Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the ultrasound image data. The processing performed by the exam analysis processor 140 view recognition deep neural network (e.g., convolutional neural network) may identify image views of an anatomical structure in ultrasound image data with a high degree of probability.

The exam analysis processor 140 may be configured to provide any identified inadequate and/or missing image views to an exam reporting processor 150 of the signal processor 132.

The signal processor 132 may include an exam reporting processor 150 that comprises suitable logic, circuitry, interfaces and/or code that may be operable to generate a summary ultrasound image examination report based on any identified inadequate and/or missing image views provided by the exam analysis processor 140. In various embodiments, the ultrasound image examination report may include a recommendation regarding whether to acquire additional images and/or a recommendation identifying additional image views to consider acquiring. The generated summary ultrasound image examination report may be presented at the display system 134. The exam reporting processor 150 may present options for confirming the end of the ultrasound examination or reopening the ultrasound examination to acquire additional images.

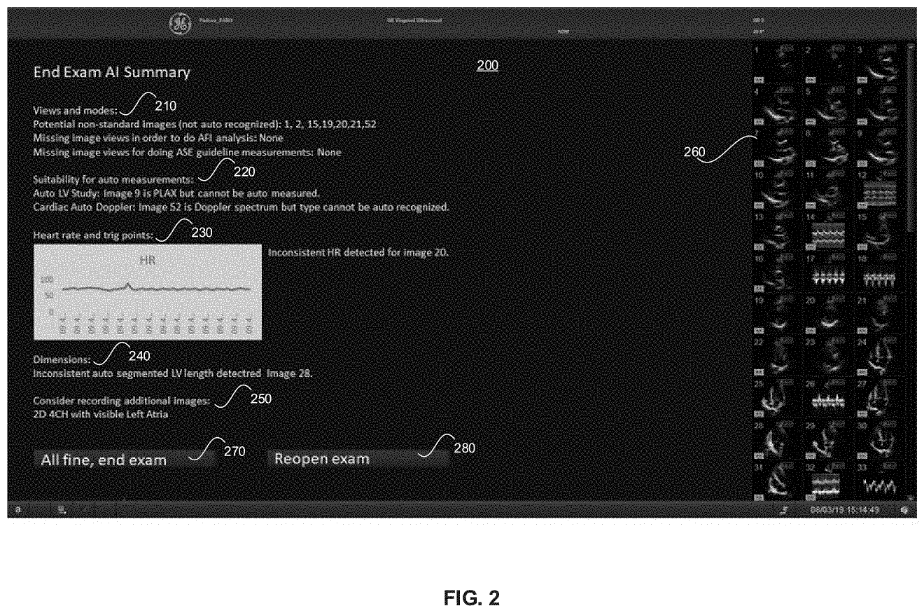

FIG. 2 is a display of an exemplary ultrasound image examination report 200 presented at the end of an ultrasound examination, in accordance with various embodiments. Referring to FIG. 2, the displayed report 200 may include a summary 210-240 of inadequate and/or missing image views, a recommendation 250, a list or set of thumbnail images of the acquired ultrasound images 260, an option for ending the examination 270, and/or an option for reopening the examination 280, among other things. In various embodiments, the summary 210-240 of inadequate and/or missing views may include a views and modes summary 210, a suitability for automated measurements summary 220, a heart rate and trig points summary 230, and a dimensions summary 240. The views and modes summary 210 may identify, for example, potential non-standard images (e.g., unrecognized views), missing image views in order to perform AFI analysis, and/or missing image views in order to perform ASE guideline measurements, among other things. The suitability for automated measurements summary 220 may identify, for example, inadequate images for performing automated measurements associated with an automated left ventricle (LV) study, a cardiac automated Doppler measurement, or the like. The heart rate and trig points summary 230 may identify inconsistent heart rate between images, which may be illustrated in graph format, for example. The dimensions summary 240 may identify inconsistent anatomical structure dimensions, such as an inconsistent left ventricle length detected in an automatically segmented image.

The recommendation summary 250 may provide a recommendation for whether to acquire additional images and/or a recommendation identifying additional image views to consider acquiring. For example, the exemplary ultrasound image examination report 200 includes a recommendation 250 to consider recording an additional two-dimensional (2D) four-chamber (4CH) view with visible left atria. Each image in the list or set of thumbnail images of the acquired ultrasound images 260 may be selectable to view a full-size image corresponding with the listed or thumbnail image. The option for ending the examination 270 and the option for reopening the examination 280 may be selectable buttons, drop down menu options, or any suitable selectable mechanism.

The ultrasound image examination report 200 may be presented at the display system 134 of FIG. 1, or any suitable display. Referring again to FIG. 1, the display system 134 may be any device capable of communicating visual information to a user. For example, a display system 134 may include a liquid crystal display, a light emitting diode display, and/or any suitable display or displays. The display system 134 can be operable to present ultrasound images, an ultrasound image examination report 200, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores medical image data, examination analysis instructions, and examination report generation instructions, for example.

Still referring to FIG. 1, the training engine 160 may comprise suitable logic, circuitry, interfaces and/or code that may be operable to train the neurons of the deep neural network(s) of the exam analysis processor 140. For example, an artificial intelligence view recognition analysis module or algorithm of the exam analysis processor 140 may be trained to automatically identify views of an anatomical structure provided in an ultrasound scan plane. For example, the training engine 160 may train the deep neural networks of the exam analysis processor 140 using databases(s) of classified scan planes. As an example, an exam analysis processor 140 may be trained by the training engine 160 with scan planes of particular views of particular anatomical structures to train the artificial intelligence view recognition analysis module or algorithm with respect to the characteristics of the particular view of the anatomical structure, such as the appearance of structure edges, the appearance of structure shapes based on the edges, the positions of the shapes relative to landmarks in the ultrasound image data, and the like. In an exemplary embodiment, the anatomical structure may be a heart and the image views may include, among other things, a 4CH view, a 2CH view, an APLAX view, a PLAX view, a SAX-AP view, a SAX-PM view, a SAX-MV view, and/or any suitable view of the heart. The structural information may include information regarding the edges, shapes, and positions of ventricles, atria, papillary muscles, inferior wall, mitral valve, apex, septum, and/or the like. In various embodiments, the databases of training scan planes may be stored in the archive 138 or any suitable data storage medium. In certain embodiments, the training engine 160 and/or training image databases may be external system(s) communicatively coupled via a wired or wireless connection to the ultrasound system 100.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

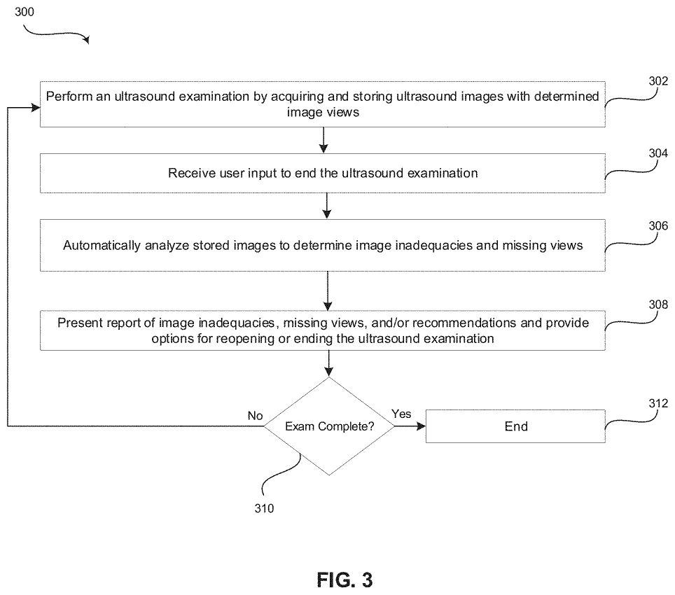

FIG. 3 is a flow chart 300 illustrating exemplary steps 302-312 that may be utilized for performing an ultrasound examination and analyzing and reporting image inadequacies and missing views at the end of the ultrasound examination, in accordance with various embodiments. Referring to FIG. 3, there is shown a flow chart 300 comprising exemplary steps 302 through 312. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 302, an ultrasound system 100 performs an ultrasound examination by acquiring and storing ultrasound images with determined image views. For example, the ultrasound system 100 may acquire images with an ultrasound probe 104 positioned at a scan position over region of interest. An ultrasound operator may record or freeze, via a user input device 130, one or more of the acquired ultrasound images. A signal processor 132 of the ultrasound system 100 may determine the image view of each of the recorded or frozen images and may store each of the images with the determined image view in archive 138 and/or in any suitable data storage medium.

At step 304, the ultrasound system 100 receives a user input to end the ultrasound examination. For example, the user input device 130 of the ultrasound system 100 may include an end exam button, or any suitable user input mechanism, selectable by an ultrasound operator to end the ultrasound examination.

At step 306, a signal processor 132 of the ultrasound system 100 may process the stored ultrasound images to detect whether the stored images include inadequacies and missing views. For example, an exam analysis processor 140 of the signal processor 132 may receive the ultrasound images acquired by probe 104 at step 302. The exam analysis processor 140 may include image analysis algorithms, one or more deep neural networks (e.g., a convolutional neural network) and/or may utilize any suitable form of image analysis techniques or machine learning processing functionality configured to automatically identify inadequate images and missing views of an anatomical structure provided in the stored ultrasound images. As an example, the exam analysis processor 140 may perform view recognition, spectrum recognition, end diastole estimation, segmentation, automated measurements (e.g., left ventricle (LV) study, cardiac automated Doppler, etc.), automated clinical findings (e.g., diastology assessment), and/or any suitable missing view analysis mechanism and image adequacy analysis mechanism. The exam analysis processor 140 may be configured to identify images having inadequacies such as, non-standard images (e.g., not automatically recognized), images that cannot be automatically measured, a varying heartbeat during a recorded image, images having inconsistent structural dimensions, and/or any suitable image inadequacy. The exam analysis processor 140 may be configured to identify missing views, such as views for performing AFI analysis, ASE guideline measurements, or the like.

At step 308, the signal processor 132 of the ultrasound system 100 may present a report 200 of image inadequacies, missing views, and/or recommendations and provide options for reopening or ending the ultrasound examination. For example, an exam reporting processor 150 of the signal processor 132 may receive the image inadequacies and missing views from the exam analysis processor 140 and may generate an ultrasound image examination report 200. The ultrasound image examination report 200 may include a summary 210-240 of inadequate and/or missing image views, a recommendation 250, a list or set of thumbnail images of the acquired ultrasound images 260, an option for ending the examination 270, an option for reopening the examination 280, and the like. The summary 210-240 may identify each inadequate image and any missing views. The recommendation 250 may provide a recommendation for whether to acquire additional images and/or a recommendation identifying additional image views to consider acquiring.

At step 310, the signal processor 132 of the ultrasound system 100 determines whether the ultrasound examination is complete. For example, the signal processor 132 may receive an operator selection, via user input device 130, to either reopen the ultrasound examination to acquire additional image views or to end the ultrasound examination. If the signal processor 132 receives a user input selecting the option to reopen the ultrasound examination, the method may return to step 302 to continue performing the ultrasound examination by acquiring and storing ultrasound images with determined imaged views. If the signal processor 132 receives a user input selecting the option to end the ultrasound examination, the method proceeds to step 312 and the ultrasound examination ends. As an example, an operator may select to reopen the ultrasound examination or to end the ultrasound examination based on information and/or recommendations provided in the ultrasound examination summary report 200 presented at step 308.

Aspects of the present disclosure provide a method 300 and system 100 for performing an ultrasound examination, analyzing the acquired images, and reporting image inadequacies and missing views at the end of the ultrasound examination. In accordance with various embodiments, the method 300 may comprise acquiring 302, by an ultrasound system 100, a plurality of ultrasound images during an ultrasound examination, each of the plurality of ultrasound images having an image view. The method 300 may comprise receiving 304, by at least one processor 132, 140, 150, a user input to end the ultrasound examination. The method 300 may comprise automatically determining 306, with artificial intelligence 140, whether one or more images in the plurality of ultrasound images includes at least one inadequacy. The method 300 may comprise automatically determining 306, with the artificial intelligence 140, whether one or more of a plurality of desired image views are not present in the plurality of ultrasound images. The method 300 may comprise presenting 308, at a display system 134, a report 200 identifying 210-240 one or more inadequate images in the plurality of ultrasound images if the artificial intelligence determines that one or more images in the plurality of ultrasound images includes the at least one inadequacy. The report 200 may identify 210 one or more views not present in the plurality of ultrasound images if the artificial intelligence determines that one or more of the plurality of desired views are not present in the plurality of ultrasound images. The method 300 may comprise providing 308, 310, at the ultrasound system 100, a selectable reopen exam option 280 to reopen the ultrasound examination.

In an exemplary embodiment, the method 300 may comprise providing 308, 310, at the ultrasound system 100, a selectable end exam option 270 to confirm the end of the ultrasound examination. In a representative embodiment, the report 200 provides a recommendation 250 for whether to reopen the ultrasound examination. In certain embodiments, the recommendation 250 identifies one or more additional image views to acquire. In various embodiments, the plurality of desired views correspond with an imaging protocol. In an exemplary embodiment, the automatic determination of whether one or more images in the plurality of ultrasound images includes at least one inadequacy is performed by at least one neural network. In a representative embodiment, the automatic determination of whether one or more of a plurality of desired images views are not present in the plurality of ultrasound images is performed by at least one neural network. In certain embodiments, the at least one inadequacy is one or more of: a non-standard ultrasound image, an ultrasound image that cannot be automatically measured, an ultrasound image associated with a varying heartbeat, and an ultrasound image having a measured structural dimension inconsistent with the structural dimension measured in other of the plurality of ultrasound images.

Various embodiments provide a system 100 for performing an ultrasound examination, analyzing the acquired images, and reporting image inadequacies and missing views at the end of the ultrasound examination. The system 100 may comprise an ultrasound system 100, at least one processor 132,140,150, and a display system 134. The ultrasound system 100 may be configured to acquire a plurality of ultrasound images during an ultrasound examination, each of the plurality of ultrasound images having an image view. The at least one processor 132,140,150 may be configured to receive a user input to end the ultrasound examination. The at least one processor 132,140,150 may be configured to automatically determine, with artificial intelligence, whether one or more images in the plurality of ultrasound images includes at least one inadequacy. The at least one processor 132,140,150 may be configured to automatically determine, with the artificial intelligence, whether one or more of a plurality of desired image views are not present in the plurality of ultrasound images. The display system 134 may be configured to present a report 200 identifying 210-240 one or more inadequate images in the plurality of ultrasound images if the artificial intelligence determines that one or more images in the plurality of ultrasound images includes the at least one inadequacy. The report 200 may identify 210 one or more views not present in the plurality of ultrasound images if the artificial intelligence determines that one or more of the plurality of desired views are not present in the plurality of ultrasound images. The ultrasound system 100 may be configured to provide a selectable reopen exam option 280 to reopen the ultrasound examination.

In a representative embodiment, the ultrasound system 100 is configured to provide a selectable end exam option 270 to confirm the end of the ultrasound examination. In an exemplary embodiment, the report 200 provides a recommendation 250 for whether to reopen the ultrasound examination. In various embodiments, the recommendation 250 identifies one or more additional image views to acquire. In certain embodiments, the plurality of desired views correspond with an imaging protocol. In a representative embodiment, one or both of the automatic determination of whether one or more images in the plurality of ultrasound images includes at least one inadequacy and the automatic determination of whether one or more of a plurality of desired images views are not present in the plurality of ultrasound images is performed by at least one neural network. In an exemplary embodiment, the at least one inadequacy is one or more of: a non-standard ultrasound image, an ultrasound image that cannot be automatically measured, an ultrasound image associated with a varying heartbeat, and an ultrasound image having a measured structural dimension inconsistent with the structural dimension measured in other of the plurality of ultrasound images.

Certain embodiments provide a non-transitory computer readable medium having stored thereon, a computer program having at least one code section. The at least one code section is executable by a machine for causing the machine to perform steps 300. The steps 300 may comprise receiving 302 a plurality of ultrasound images during an ultrasound examination, each of the plurality of ultrasound images having an image view. The steps 300 may comprise receiving 304 a user input to end the ultrasound examination. The steps 300 may comprise automatically determining 306, with artificial intelligence, whether one or more images in the plurality of ultrasound images includes at least one inadequacy. The steps 300 may comprise automatically determining 306, with the artificial intelligence, whether one or more of a plurality of desired image views are not present in the plurality of ultrasound images. The steps 300 may comprise presenting, at a display system 134, a report 200 identifying 210-240 one or more inadequate images in the plurality of ultrasound images if the artificial intelligence determines that one or more images in the plurality of ultrasound images includes the at least one inadequacy. The report 200 may identify 210 one or more views not present in the plurality of ultrasound images if the artificial intelligence determines that one or more of the plurality of desired views are not present in the plurality of ultrasound images. The steps 300 may comprise providing 308, 310 a selectable reopen exam option 280 to reopen the ultrasound examination.

In various embodiments, he steps 300 may comprise providing 308, 310 a selectable end exam option 270 to confirm the end of the ultrasound examination. In an exemplary embodiment, the report 200 provides a recommendation 250 for whether to reopen the ultrasound examination. The recommendation 250 may identify one or more additional image views to acquire. In a representative embodiment, the plurality of desired views correspond with an imaging protocol. In certain embodiments, the at least one inadequacy is one or more of: a non-standard ultrasound image, an ultrasound image that cannot be automatically measured, an ultrasound image associated with a varying heartbeat, and an ultrasound image having a measured structural dimension inconsistent with the structural dimension measured in other of the plurality of ultrasound images.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y) }. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for performing an ultrasound examination, analyzing the acquired images, and reporting image inadequacies and missing views at the end of the ultrasound examination.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
acquiring, by an ultrasound system, a plurality of ultrasound images during an ultrasound examination, each of the plurality of ultrasound images having an image view;
receiving, by at least one processor, a user input to end the ultrasound examination;
automatically determining, with artificial intelligence, whether one or more images in the plurality of ultrasound images includes at least one inadequacy;
automatically determining, with the artificial intelligence, whether one or more of a plurality of desired image views are not present in the plurality of ultrasound images;
presenting, at a display system, a report identifying:
one or more inadequate images in the plurality of ultrasound images if the artificial intelligence determines that one or more images in the plurality of ultrasound images includes the at least one inadequacy, and
one or more views not present in the plurality of ultrasound images if the artificial intelligence determines that one or more of the plurality of desired views are not present in the plurality of ultrasound images; and
providing, at the ultrasound system, a selectable reopen exam option to reopen the ultrasound examination.

2. The method of claim 1, comprising providing, at the ultrasound system, a selectable end exam option to confirm the end of the ultrasound examination.

3. The method of claim 1, wherein the report provides a recommendation for whether to reopen the ultrasound examination.

4. The method of claim 3, wherein the recommendation identifies one or more additional image views to acquire.

5. The method of claim 1, wherein the plurality of desired views correspond with an imaging protocol.

6. The method of claim 1, wherein the automatic determination of whether one or more images in the plurality of ultrasound images includes at least one inadequacy is performed by at least one neural network.

7. The method of claim 1, wherein the automatic determination of whether one or more of a plurality of desired images views are not present in the plurality of ultrasound images is performed by at least one neural network.

8. The method of claim 1, wherein the at least one inadequacy is one or more of:
a non-standard ultrasound image,
an ultrasound image that cannot be automatically measured,
an ultrasound image associated with a varying heartbeat, and
an ultrasound image having a measured structural dimension inconsistent with the structural dimension measured in other of the plurality of ultrasound images.

9. A system comprising:
an ultrasound system configured to acquire a plurality of ultrasound images during an ultrasound examination, each of the plurality of ultrasound images having an image view;
at least one processor configured to:
receive a user input to end the ultrasound examination;
automatically determine, with artificial intelligence, whether one or more images in the plurality of ultrasound images includes at least one inadequacy;
automatically determine, with the artificial intelligence, whether one or more of a plurality of desired image views are not present in the plurality of ultrasound images; and
a display system configured to present a report identifying:
one or more inadequate images in the plurality of ultrasound images if the artificial intelligence determines that one or more images in the plurality of ultrasound images includes the at least one inadequacy, and
one or more views not present in the plurality of ultrasound images if the artificial intelligence determines that one or more of the plurality of desired views are not present in the plurality of ultrasound images,
wherein the ultrasound system is configured to provide a selectable reopen exam option to reopen the ultrasound examination.

10. The system of claim 9, wherein the ultrasound system is configured to provide a selectable end exam option to confirm the end of the ultrasound examination.

11. The system of claim 9, wherein the report provides a recommendation for whether to reopen the ultrasound examination.

12. The system of claim 11, wherein the recommendation identifies one or more additional image views to acquire.

13. The system of claim 9, wherein the plurality of desired views correspond with an imaging protocol.

14. The system of claim 9, wherein one or both of the automatic determination of whether one or more images in the plurality of ultrasound images includes at least one inadequacy and the automatic determination of whether one or more of a plurality of desired images views are not present in the plurality of ultrasound images is performed by at least one neural network.

15. The system of claim 9, wherein the at least one inadequacy is one or more of:
a non-standard ultrasound image,
an ultrasound image that cannot be automatically measured,
an ultrasound image associated with a varying heartbeat, and
an ultrasound image having a measured structural dimension inconsistent with the structural dimension measured in other of the plurality of ultrasound images.

16. A non-transitory computer readable medium having stored thereon, a computer program having at least one code section, the at least one code section being executable by a machine for causing the machine to perform steps comprising:
receiving a plurality of ultrasound images during an ultrasound examination, each of the plurality of ultrasound images having an image view;
receiving a user input to end the ultrasound examination;
automatically determining, with artificial intelligence, whether one or more images in the plurality of ultrasound images includes at least one inadequacy;
automatically determining, with the artificial intelligence, whether one or more of a plurality of desired image views are not present in the plurality of ultrasound images;
presenting, at a display system, a report identifying:
one or more inadequate images in the plurality of ultrasound images if the artificial intelligence determines that one or more images in the plurality of ultrasound images includes the at least one inadequacy, and
one or more views not present in the plurality of ultrasound images if the artificial intelligence determines that one or more of the plurality of desired views are not present in the plurality of ultrasound images; and
providing a selectable reopen exam option to reopen the ultrasound examination.

17. The non-transitory computer readable medium of claim 16, comprising providing a selectable end exam option to confirm the end of the ultrasound examination.

18. The non-transitory computer readable medium of claim 16, wherein the report provides a recommendation for whether to reopen the ultrasound examination, the recommendation identifying one or more additional image views to acquire.

19. The non-transitory computer readable medium of claim 16, wherein the plurality of desired views correspond with an imaging protocol.

20. The non-transitory computer readable medium of claim 16, wherein the at least one inadequacy is one or more of:
a non-standard ultrasound image,
an ultrasound image that cannot be automatically measured,
an ultrasound image associated with a varying heartbeat, and
an ultrasound image having a measured structural dimension inconsistent with the structural dimension measured in other of the plurality of ultrasound images.
